(19) United States
(12) Patent Application Publication
(10) Pub. No.: US 2009/0035775 A1
(43) Pub. Date: Feb. 5, 2009

(54) PCR-BASED DETECTION METHOD FOR CHLAMYDIA TREACHOMATIS

(75) Inventors: Daman Saluja, Delhi (IN); Uma Chaudhury, Delhi (IN); Mashook Ali, Delhi (IN); Poonam Sachdeva, Delhi (IN)

Correspondence Address: THE WEBB LAW FIRM, P.C., 700 KOPPERS BUILDING, 436 SEVENTH AVENUE, PITTSBURGH, PA, 15219, US

(73) Assignees: UNIVERSITY OF DELHI, New Delhi (IN); THE SECRETARY, DEPARTMENT OF BIOTECHNOLOGY, New Delhi (IN)

(21) Appl. No.: 12/064,143

(22) PCT Filed: Aug. 7, 2006
(86) PCT No.: PCT/IN2006/000282

§ 371 Date: Sep. 17, 2008

(30) Foreign Application Priority Data
Aug. 17, 2005 (IN) 1589DEL/2005

(51) Int. Cl. C12Q 1/68

(52) U.S. Cl. 435/6

(57) ABSTRACT

A process for designing of PCR-based detection method for Chlamydia trachomatis comprising designing of PCR primers from the genome sequence of Chlamydia trachomatis selecting DNA sequence of genes for PCR based diagnostic of Chlamydia trachomatis, optimizing the PCR conditions of the PCR primers; subjecting the said genes and primers to the step of characterization.

This invention relates to a designing of PCR based detection method for chlamydia trachomatis.

### BACKGROUND OF THIS INVENTION

Culture method: Chlamydiae were first detected by light microscopy in conjuctival scrapings from orangutangs inoculated with material from trachoma patients in 1907. The diagnostic sensitivity and specificity of light microscopy, however, are not satisfactory. Because Chlamydia depends on ATP and other nutritional factors from a host cell it can reproduce only in other cell. The inclusion body, containing thousands of C. trachomatis can be visualized by staining with fluorescein-conjugated antibody directed against one of the organism's surface antigens. Because the inclusion body is highly characteristic, cell culture is considered to have a specificity of 100%.

Sensitivity of this method is as low as 50% as organisms may lose infectivity during transportation and storage, which will reduce the likelihood of propagation. In addition, the surface area of the cell culture layer and/or the amount of sample material added to the cell culture influence the sensitivity. Cell culture, however, is time-consuming, laborious and expensive and can therefore be provided by only a few central laboratories.

Antigen detection: Antigen detection methods comprise Enzyme-inked immunosorbent assays (ELISA) and Direct immunofluorescence assays (DFA). The currently commercially available ELISA all use the LPS as antigen. The LPS part of Chlamydia binds to immobilized anti-LPS antibodies and the ELISA tests are therefore genus specific and detect all Chlamydia species. A secondary antibody that is bound to the Chlamydia is linked to an enzyme, which generates a colour change, measured as optical density, on addition of subtrate. In DFA, fluorescein-conjugated antibodies directed against either the LPS or the MOMP component react with the Chlamydia surface. The fluorescein can subsequently be visualized by fluorescence microscopy.

The diagnostic efficacy of these methods is not high enough to warrant clinical use unless the need for a fast result overweighs the lower diagnostic accuracy. Also, the ELSA tests may reveal positive results in the presence of other organisms such as E. coli and Bacteroides sp, and Staphylococcus aureus may be captured instead of Chlamydia due to binding to the Fc region of the antibodies, thereby causing false-positive reactions. DFA requires skilled personnel in order to differentiate C. trachomatis organisms from non-specific fluorescent particles.

DNA/RNA detection: DNA/RNA detection assays can be divided into probe assays and amplification assays. In probe assays a synthesized single stranded oligonucleotide hybridizes to a part of C. trachomatis DNA or RNA. The most widely used probe technique is the Gen-Probe assay, in which a probe reacts with ribosomal RNA (rRNA) of C. trachomatis, which is present in hundreds of copies in each organism.

The diagnostic performance of non-amplified probe technique is not substantially different from that of the best ELISA.

Nucleic acid amplification tests (NAATs): In nucleic acid amplification Tests specific probes hybridize to C. trachomatis DNA or RNA and the DNA/RNA flanked by the primers (target DNA) is exponentially copied. Target gene—The Plasmid: The plasmid is unique for C. trachomatis, is well conserved within the species, and is present in approxdmaterly 10 copies in each C. trachomatis organism. Using the plasmid as target DNA should therefore theoretically lower the detection limit by a factor of 10 compared with a single chromosomal gene, for example MOMP gene.

The 16S-rRNA gene: By using the 16S-rRNA gene, which is present in all bacteria and is the most conserved gene known, all Chlamydia species can be detected by just one primer set. This is done by constructing the primers to anneal at the genus-specific regions of the 16S-rRNA gene. The genus specific regions flank variable-regions that are specific for each species. The amplified products the comprise the variable region flanked by the two genus specific regions. The species can be determined by specific probe or RFLP, or by DNA sequencing.

Target gene for NAATs Target gene for NAATs The Plasmid: Some studies give evidence or suggest that the plasmid-free variants are present in clinical samples, and although it may seem that plasmid is involved in DNA replication, it has been possible to culture a plasmid-free variant. Thus, the infections caused by plasmid-free variants will be undetected if the plasmid is used as target gene.

The 16S-rRNA gene: Due to high homology of the 16S RNA gene with other organisms, optimal reaction conditions are crucial in order to avoid annealing of primers to 16S-rRNA gene of the other organisms that are present in all non-sterile clinical samples.

### OBJECTS OF THE INVENTION

An object of this invention is to propose a designing of PCR-based detection method for Chlamydia trachomatis.

Another object of this invention is to propose identfication of unique sequences in the genome of C. trachomatis.

Further object of this invention is to propose a designing of PCR-based diagnostic method for chlamydia trachomatis.

Still further object of this invention is to propose a multplex PCR based detection method for Chlamydia trachomatis and Neisseria gonorrhoeae.

### BRIEF DESCRIPTION OF THIS INVENTION

According to this invention there is provided a process for designing of PCR-based detection method for chlamydia trachomatis comprising designing of PCR primers from the genome sequence of C. trachomatis selecting DNA sequence of genes for PCR based diagnostic of C. trachomatis, optimizing the PCR conditions of the PCR primers; subjecting the said genes and primers to the step of characterization.

### DETAILED DESCRIPTION OF THIS INVENTION

### Designing of PCR Primers:

The key to any polymerase chain reaction is a pair of oligonucleotide primers of defined sequence. Primers were designed using the GENEFISHER program from the web site www.ebl.ac.uk. From the genome sequence of C. trachomatis available on the medline, the DNA sequence of those genes, which appear to be unique to C. trachomatis, were selected using Blast. For C. trachomatis DNA sequences of two genes were selected for PCR based diagnostic method. These were gyrA genes, phospholipase D endonuclease superfamily gene (PRPHA).

### Optimization of the PCR Conditions:

For initial standardization of our PCR assays to be used in a clinical setting, various experiments were performed with authentic DNA as template and the data was confirmed by using the patient samples.

### Characterization of the PCR Amplified Product:

The authenticity of the amplified products was established by sequencing of the amplified products. The amplified gene products were purified from the gel, sequenced and nucleotide sequence obtained was matched with that of the known DNA sequence in the Public domain. Complete identity in sequence was observed for both amplified products with their respective genes.

Comparison of the Specificity of the PCR Assays Using the GyrA Primers for Chlamydia trachomatis:

In order to select one or two primer pair set that could be used routinely for pre-clinical evaluation we tested the specificity of each primer pair. To evaluate the specificity of the selected primers, DNA extracted from several positive clinical isolates of Chlamydia trachomatis, six non-gonococcal Neisseria species, three genital commensals and four N. gonorrhoeae isolates as well as C. albicans, C. glabrata, Herpes simplex, Mycoplasma etc. were used as templates.

Development of a Multiplex-PCR Assay to Detect C. trachomatis:

To increase the specificity our PCR based diagnostic system we developed multiplex PCR assay using gyrA and PHA primers. Initial standardization for multiplex PCR was done using authentic DNA as well as with known positive samples. The method was then extended to the clinical samples. All assays were compared with simple PCR for the two genes separately. Concentration of dNTPs used was 300 μM, Taq DNA polymerase was used 1.25 U. First ten cycles were run with gyrA primers and then PHA primers were added for the following 28 cycles.

The designed primers will be used for the diagnosis of Chlamydia trachomatis in patient samples. Further, multiplex PCR has been developed for Chlamydia trachomatis and Neisseria gonorrhoeae, another STD causing organism which co infects the patients. Presently Various other methods like ELISA, DFA, Culture have been used for the diagnosis of Chlamydia trachomatis which are not very sensitive as compared to PCR. PCR of 517 bp region of plasmid DNA have been used for the diagnosis of Chlamydia trachomatis, but it is not of much advantage for the diagnosis, as plasmid is not found In all the strains of Chlamydia trachomatis, so it cannot be detected in the samples, which are Chlamydia positive but lacking plasmid.

### EXAMPLE 1

### Standardisation of Large Scale Collection of Test Sample

### Processing of Clinical Specimens (Urethral Swabs) for DNA Amplification:

The endogenous inhibitors present in the clinical sample that can interfere with nucleic acid amplification influence the performance of PCR testing with the urogenital specimens. A transport medium/collection medium was designed that would remove or neutralize the inhibitors present in the clinical sample. This also helped in the collection of large number of samples.

400 μl sample of specimen was centrifuged for 30 min at 14,000×g, and the pellet was treated with 40 μl of lysis buffer (50 mM Tris-HCl [pH 7.5], 1% Triton X-100, 1 mM EDTA, 400 μg of proteinase K per ml). After incubation at 37° C. for 1 h, the lysates were boiled for 10 min and centrifuged briefly. From each lysate, 10 μl was added to 40 μl of PCR mixture.

Detection of Chlamydia trachomatis Using PCR Based Assays:

We have designed two pairs of primers based on the sequence of Chlamydia trachomatis available on net. The PCR conditions using each primer pair have been standardized. In order to select one or two primer pair set that could be used for pre-clinical evaluation it is necessary to test the specificity of each primer pair. This is necessary so as to avoid false negatives. We have standardized gyrA primer to be used for clinical evaluation. Second confirmatory test is being done using PRPHA primer or multiplex PCR using both the sets of primers.

### EXAMPLE 2

### Development of a Multiplex-PCR Assay for Simultaneous Detection of N. gonorrhoeae and C. trachomatis in the Urogenital Specimens

Since Chlamydia trachomatis often co-infects with N. gonorrhoeae and signs and symptoms of the disease are similar to that of gonorrhoea. To check for this coinfecting microorganism in the clinical specimens, we performed a multiplex PCR assay to detect the two organisms simultaneously. For this multiplex PCR assay, the 23S rRNA gene was selected for detecting N. gonorrhoeae and the gyrA gene of the C. trachomatis was used as the target. Conditions for the multiplex-PCR were standardized after testing the samples individually and then in a multiplex assay using authentic clinical samples. When tested individually for N. gonorrhoeae and C. trachomatis, out of the 225 specimens tested, only 17 tested positive for C. trachomatis and 204 tested positive for N. gonorrhoeae (see Table 2). Similar results were obtained using the multiplex PCR assay. Of the seventeen specimens, which were found positive for C. trachomatis, three specimens were also infected with N. gonorrhoeae. It is pertinent to mention that the conditions for multiplex PCR need to be standardized separately as the primers may not amplify with same efficiency when tested together than when tested separately. Moreover, the two co infecting organisms may not be present in the same amount. We found that the concentrations of the four dNTPs when increased from 200 to 300 μM and when the amount of Taq DNA polymerase was increased from 1.5 U to 3 U, better multiplex-PCR results were obtained. Ten cycles were first run with the primer set CT2A and CT2B to increase the number of available templates and hence competitiveness for the subsequent cycles. This was then followed by a further thirty cycles after the addition of the 23S rRNA primer set.

TABLE 2
Comparison of single and Multiplex-PCR results for the detection of
Chlamydia trachomatis and Neisseria gonorrhoeae in 225 urogenital specimens.

Results

| Specimen | C.trachomatis- | | Multiplex-PCR | |
|---|---|---|---|---|
| # | PCR | N.gonorrhoeae-PCR | C.trachomatis-PCR | N-gonorrhoeae-PCR |
| 201 | − | + | − | + |
| 7 | − | − | − | − |
| Multi- | | | | |
| plexing | | | | |
| 14 | + | − | + | − |
| 3 | + | + | + | + |

1-5. (canceled)

6: A process for designing of PCR-based detection method for Chlamydia trachomatis comprising the steps of:
designing of a pair of oligonucleotide primers of define sequence from the genome sequence of Chlamydia trachomatis selecting DNA sequence of genes for PCR-based diagnostic of Chlamydia trachomatis;
optimizing the PCR conditions of the PCR primers; and
subjecting the said genes and primers to the step of characterization.

7: The process as claimed in claim 6, wherein said genes are gyrA genes of 462 bp and phospholipase D endonuclease genes of 368 bp.

8: The process as claimed in claim 6, further comprising the step of authenticating said genes and primers by sequencing the said amplified products.

9: A set of PCR primers for PCR-based detection of Chlamydia trachomatis comprising a first oligonucleotide primer having a sequence substantially homologous to a first portion of a gyrA gene, a second oligonucleotide primer having a sequence substantially homologous to a second portion of the gyrA gene.

10: The set of PCR primers for PCR-based detection of Chlamydia trachomatis as recited in claim 9, further comprising a third oligonucleotide primer having a sequence substantially homologous to a first portion of a phospholipase D endonuclease gene, and a fourth oligonucleotide primer having a sequence substantially homologous to a second portion of the phospholipase D endonuclease gene.

11: The set of PCR primers for PCR-based detection of Chlamydia trachomatis as recited in claim 9, wherein the first portion of the gyrA gene begins at or about a 3′ end of a sense strand of the gyrA gene, and the second portion of the gyrA gene terminates at or about a 5′ end of the sense strand of the gyrA gene.

12: The set of PCR primers for PCR-based detection of Chlamydia trachomatis as recited in claim 10, wherein the first portion of the phospholipase D endonuclease gene is at or about a 3′ end of a sense strand of the phospholipase D endonuclease gene, and the second portion of the phospholipase D endonuclease gene is at or about a 5′ end of the sense strand of the phospholipase D endonuclease gene.
